SEQUENCE LISTING (2 sequences)

SEQ ID NO: 1 - protein, 16 aa, Artificial Sequence
  It is synthesized.
  misc_feature (2)..(2): Xaa is Diazirine-Lys
  misc_feature (14)..(14): Xaa is Arg(me2a)
CXKAKTGAAGKFKXGK

SEQ ID NO: 2 - protein, 16 aa, Artificial Sequence
  It is synthesized.
  misc_feature (2)..(2): Xaa is Diazirine-Lys
CXKAKTGAAGKFKRGK

(19) United States
(12) Patent Application Publication
(10) Pub. No.: US 2023/0251265 A1
(43) Pub. Date: Aug. 10, 2023

(54) PREPARATION METHOD AND THE APPLICATION OF CAPTURE MAGNETIC BEAD TARGETING WEAK PROTEIN-PROTEIN INTERACTIONS BASED ON THE PHOTO-AFFINITY COVALENT LINKAGE STRATEGY

(71) Applicant: NANJING NORMAL UNIVERSITY, Nanjing (CN)

(72) Inventor: Tao GAO, Nanjing (CN)

(21) Appl. No.: 17/780,821

(22) PCT Filed: Sep. 23, 2020
(86) PCT No.: PCT/CN2020/116950

§ 371 Date: Dec. 25, 2022

(30) Foreign Application Priority Data
Dec. 6, 2019 (CN) 201911247058.5

(51) Int. Cl. G01N 33/68; C07K 17/14; C07K 17/08; G01N 33/543

(52) U.S. Cl. CPC G01N 33/6845; C07K 17/08; C07K 17/14; G01N 33/6848; G01N 33/54326; G01N 2446/00

(57) ABSTRACT

A magnetic bead is respectively modified by two functional molecular layers from the inside out, termed as the polyethylene glycol (PEG) passivation layer and the photo-affinity peptide probe layer, respectively; PEG passivation layer is introduced at the surface of a magnetic bead, forming a PEG-modified magnetic bead, and the photo-affinity peptide probe layer is a molecular layer of peptide whose N-terminal end is modified with the thiol group and the diazirine group; the PEG passivation layer on the capture magnetic bead is used to reduce non-specific interaction of protein molecules, while the photo-affinity peptide probe layer can specifically recognize and capture target proteins; the weak interaction between the photo-affinity peptide probe and target proteins is converted to covalent linkage under the UV irradiation, thus achieving specific and efficient capture and magnetic separation of interacted proteins.

### INCORPORATION BY REFERENCE OF SEQUENCE LISTING

The Sequence Listing in the ASCII text file, named as 220581_ST25.txt, and submitted to the United States Patent and Trademark Office via EFS-Web, is incorporated herein in the invention.

### FIELD OF THE INVENTION

The present invention relates to the technical of protein-protein interaction analysis, and specifically relates to a preparation method and the application of a kind of capture magnetic bead that is able to target weak protein-protein interactions based on the photo-affinity covalent linkage.

### BACKGROUND OF THE INVENTION

Protein-protein interactions (PPIs) mediate almost all life activities at the molecular level. Identification of PPIs is critical for the biomedical research, enabling the discovery of new signal pathways, the study of disease mechanisms, the identification of new drug targets, and the design of biomedical sensors, etc. As protein molecules are the direct bearers of biological functions, it has become a primary method to explore the molecular mechanism by discovering the unknown mode of action in the signal transduction pathway through known protein molecules. PPIs technology is an indispensable tool for biomedical research. Currently, the techniques for PPIs′ analysis include the pull-down assay (Pull-down assay), co-immunoprecipitation assay (Co-IP), tandem affinity purification-mass spectrometry (TAP-MS), yeast two-hybrid system, bioinformatics analysis, etc. However, taking the complexity of PPIs into consideration, the analysis of PPIs still has great technical challenges.

Based on the side chains of amino acids and the structural motifs of proteins, there are at least six interaction modes are known, including (1) intra-domain interaction: the reaction interface is located within the same structural domain; (2) inter-domain interaction: the reaction interface is located within two structural domains of the same peptide chain; (3) homo-oligomers interaction: the reaction interface is located within the same peptide chain of permanent interaction; (4) homo-complexes interaction: the reaction interface is located within the same peptide chain of transient interaction; (5) hetero-oligomers interaction: the reaction interface is located within different peptide chains of permanent interaction; (6) hetero-complexes interaction: the reaction interface is located within different peptide chains of transient interaction. In addition to the dynamic changes in mode of action and the combined effects that influence and determine life activities, the kinetics, thermodynamics, stoichiometry, and cofactors can affect PPIs. Therefore, the appropriate detection method should be selected according to the different modes of action. For example, the Co-IP assay is the most commonly used method to identify PPIs, which relies on the antibody affinity for the decoy protein, so as to capture the candidate proteins or protein complexes, followed by the identification of captured proteins with mass spectrometry, or western blotting. However, the applicability of the Co-IP method is limited to screen high-abundance/high-affinity binding proteins and is not suitable for screening low-affinity binding proteins mediated by transient and weak PPIs. Moreover, the time-consuming and complex operational steps between sample preparation and detection can create uncertainty in analytical results. At the same time, the instability of antibody can also lead to difference of results.

Post-translational modifications (PTMs), known as the chemical modifications at the sub-molecular level of the protein, represents the primary mechanism for mediating and regulating PPIs. It has been demonstrated by more and more researches that PTMs-mediated PPIs plays an important role in the dynamic biochemical reactions of cells. Screening the PTMs sites and elucidating the functions of PTMs are therefore crucial in biomedical studies. Presently, although many techniques can find PTMs sites on proteins, identifying the PTMs-interacted proteins remains challenging because the sub-molecular modifications on a protein tends to be low-abundant, dynamic, and mediate weak transient interactions effect. The available approaches cannot meet the requirements of the PTMs-mediated PPIs assay.

### SUMMARY

Purpose of the invention: Aiming at the problems existing in the current technology, the present invention relates to a weak protein-protein interaction capture magnetic bead based on photo-affinity covalent linkage, termed as photo-affinity magnetic beads (PAMBs). The invention proposed to fabricate photo-affinity magnetic beads based on photo-affinity linkage technology, and applied to protein capture systems for the first time, achieving the transformation of “protein-protein” weak interaction into covalent linkage, which provides a magnetic bead and new capture method for the analysis of PPIs mediated by weak protein interactions (e.g., post-translational modifications, PTMs), and can be effectively used in the universal assay of identifying PTMs-mediated PPIs.

The invention also provides a preparation method and application of a weak protein-protein capture magnetic bead based on photo-affinity covalent linkage.

Technical Proposal: To achieve the above purpose of the invention, as described in the invention, a kind of capture magnetic bead that is able to targeting weak protein-protein interactions, the described magnetic bead is respectively modified by two functional molecular layers from the inside out: the polyethylene glycol PEG passivation layer and the photo-affinity peptide probe layer, said polyethylene glycol PEG passivation layer (a PEG molecule) is introduced at the surface of a magnetic bead, forming a PEG-modified magnetic bead; said photo-affinity peptide probe layer is a peptide with a thiol group and diazirine at the N-terminal.

Wherein, two reactive groups on the peptide probe are the thiol group on the side chain of cysteine and the photo-reactive group diazirine modified on the ε-NH₂ of the lysine.

Wherein, the PEG in the said polyethylene glycol PEG passivation layer is a PEG molecule respectively modified with the amino group and the thiol group at both ends, namely NH2-PEG-SH, for forming the passivation layer, and serving as an intermediate linking molecule to connect magnetic beads and photo-affinity peptide probes.

Wherein, said photo-affinity peptide probe layer is a synthesized peptide with two reactive groups modified at the N-terminal, the thiol group on the side chain of cysteine and the photo-reactive group diazirine modified on the ε-NH₂ of the lysine.

Said photo-affinity peptide sequence is: N-terminal modified cysteine Cys, followed by a diazirine modified lysine Lys, followed by a peptide capable of targeting interacted proteins, pattern sequence is: CK (Diazirine)(x)ₙ, the X represents an amino acid or a post-translational modified amino acid, n stands for the number of amino acids.

P2: C-K (Diazirine)-KAKTGAAGKFKR (Me2a) GK (SEQ ID NO.: 01) .

As an optimization, the sequence of the photo-affinity peptide probes is:

P2: C-K (Diazirine)-KAKTGAAGKFKR (Me2a) GK (SEQ ID NO.: 01).

The preparation method of the weak protein-protein interaction capture magnetic beads based on photo-affinity covalent linkage as the invention said, comprising the following steps:

The PEG passivation layer is introduced at the surface of a magnetic bead, forming a PEG-modified magnetic bead:

The solution of NH2-PEG5000-SH and the NHS activated magnetic beads solution were equilibrated to room temperature. Specifically, 30 µL of magnetic beads were taken and placed on a magnetic stand, discarded the supernatant, then gently vortexed for 15 s after adding pre-cooled 1 mM glacial acetic acid solution, collected the beads and added 300 µL NH2-PEG5000-SH solution immediately, then vortexed for 30 s. The solution was incubated with rotation for 2 h at room temperature. The magnetic beads were collected after washing two times with 1 mL of 0.1 M glycine (pH 2.0) and then by ultrapure water respectively, and then stored at 4° C. before use.

(2) Synthesis of photo-affinity peptide probes: According to the pre-designed peptide sequence (such as P2 sequence), the carboxyl group of the C-terminal amino acid of the peptide chain to be synthesized was covalently fixed to an insoluble polymer resin, and then the repeatedly deprotected from the C-terminal to the N-terminal (remove the protective group on the α-amino group of the amino acid to be linked). Follow by activating (activate the carboxyl group of the previous amino acid already attached to the solid column), coupling (allow condensation between the deprotected amino group and the activated carboxyl group to form a peptide bond), washing and filtrating (remove various reagents that have not been reacted), until the desired synthesis of the peptide chain is achieved. The peptide with diazirine label was achieved by the reaction of the succunimidyl ester-activated diazirine with the amino group of the lysine side chain at the end of the peptide chain. The photo-affinity peptide probe was obtained by cleavage and separation from the resin.

(3) Preparation of photo-affinity capture magnetic beads: Prepare the photo-affinity peptide probe solution, immediately add the PEG-mediated beads in step 1, and mix well, collected magnetic beads were incubated with shaking at room temperature and collected, stored the obtained capture magnetic beads (PAMBs) for later use .

As a optimization, wherein said in step 1, the solution of NH₂-PEG5000-SH and magnetic beads were equilibrated to room temperature, the beads were placed on the magnetic stand and discarded the supernatant, then gently vortexed after adding glacial acetic acid solution, collected the beads and immediately proceeded by adding NH2-PEG5000-SH solution (PEG), then vortexed, the solution was incubated with rotation at room temperature, the PEG-modified beads were collected after washing, and then stored at 4° C. for later use.

As an optimization, wherein said in step 2, the polymer resin is Fmoc-Lys (Boc)-Wang resin.

Further, wherein said in step 2, Fmoc-Lys (Boc)-Wang resin (substitution value, 0.35 mM g⁻¹) was used as the starting material, and peptides was synthesized from the C terminal to the N terminal by the Fmoc solid-phase method. Specifically, a volume ratio of 25% hexahydropyridine (dissolved in N,N′-dimethylformamide (DMF)) was used to remove the N-terminal Fmoc protecting group and make the N-terminus a free amino group, then used the amino acid raw material of 3 times the volume of resin to introduce the second end of the C-terminus. The individual amino acid residues were linked in turn to complete the synthesis of the entire peptide. The peptide with diazirine label was achieved by direct reaction of the diazirine-activated ester with a specific site lysine side chain amino group. After finishing each step of the above reaction, the resin was washed with DMF more than six times and the reaction was controlled by Kaiser Test. If the condensation reaction of an amino acid was incomplete, the condensation would be repeated once until the desired target peptide was obtained. Since diazirine was unstable under light, subsequent operations must be carried out under dark conditions. The target peptide was cleaved from the resin using a 90% trifluoroacetic acid (TFA) cleavage reagent and removed the side chain protecting group at 30° C. for 3 h. A large amount of pre-cooled anhydrous ether was added to the filtrate to precipitate the peptide by centrifugating. After washing repeatedly with diethyl ether anhydrous, it was dried to obtain a crude peptide. The crude peptide was purified by reversed-phase high performance liquid chromatography (HPLC). Mobile phase A was aqueous solution containing 0.05% TFA and 2% acetonitrile (CAN), mobile phase B was 90% acetonitrile/water, flow rate was 25 mL min⁻¹. After lyophilizing the solvent, a pure peptide in a fluffy state was obtained, which is a photo-affinity peptide probe. The purity was determined by MALDI-TOF.

Wherein, the photo-affinity peptide probe solution said in step 3 uses dimethyl sulfoxide as a solvent, and the ratio of the peptide probe solution to the initial bead solution volume used to form the PEG-modified magnetic bead is 10:1.

In which step 3, the solution of photo-affinity peptide probes and PEG-modified magnetic beads were incubated at room temperature. Under the reducing condition of Dimethyl sulfoxide, the disulfide bonds were formed, and photo-affinity peptide probes were linked to the magnetic beads. The capture beads were obtained after washing with PBS and stored at 4° C.

Specific method: The ligation of peptide in step 3, firstly prepare 0.5 mg mL⁻¹ photo-affinity peptide probe solution (40% DMSO aqueous solution as the solvent), immediately add the PEG-modified magnetic beads of step 1 and mix well, The mixtures were incubated for 4 h at room temperature by using Shaker at 200 rpm. Add 1 mL of Storage Buffer to the beads and mix, collect magnetic beads with a magnetic stand, discard the supernatant and wash twice, add 300 µL of Storage Buffer, and store the obtained capture magnetic beads (PAMBs) at 4° C. for later use.

Mentioned Wash Buffer A is 1 mM glacial acetic acid; Coupling Buffer is 50 mM borate pH 8.5 and 50 mM PB; NH₂ -PEG5000-SH solution is the reagent with 0.1 M concentration; Wash Buffer B is 0.1 M glycine pH 2.0; Storage Buffer is the Coupling Buffer containing mass fraction 0.05% sodium azide in Coupling Buffer.

Applications of the weak protein-protein interaction capture magnetic bead based on photo-affinity covalent linkage said in the invention in the capture of weak interaction proteins.

Applications, said the invention, added a protein sample to magnetic beads, gently vortexed at room temperature, and simultaneously irradiated with an ultraviolet lamp, to activate covalent linkage between diazirine and captured protein, then collected and washed, the protein molecules linked to the magnetic beads is the captured protein molecules, added reducing reagent to cleave the disulfide bonds between NH2-PEG-SH and photo-affinity peptide probe, released the photo-affinity peptide probe bound with the target protein molecules, separated and identified the captured protein molecules by polyacrylamide gel electrophoresis.

The invention prepared the capture magnetic bead targeting weak protein-protein interactions for the capture and analysis of PTM-mediated protein molecules. The present invention uses photo-affinity technique and proposes a weak protein-protein interaction based on the principle of photo-affinity technique, which achieves the conversion of weak interaction into stable covalent linkage, and based on which capture magnetic beads PAMBs are prepared for the analysis and identification of PPIs mediated by PTMs or other weak interactions. In addition, the method is simple to operate, saves time, and has high specificity for the detection of PTMs-mediated weak, transient PPIs.

In the invention, PAMBs consist of magnetic beads, polyethylene glycol (PEG) passivation layer and photo-affinity peptide probes, as shown in FIG. 1. Magnetic beads refer to NHS-activated magnetic beads; PEG passivation layer is PEG with terminal modified thiol; two reactive groups get modified at the N-terminal of the photo-affinity peptide probe, the thiol group on the side chain of cysteine (C) and the photo-reactive group (diazirine) modified on the ε-NH₂ of the lysine. The thiol group can link to the PEG terminal thiol group by forming disulfide bond, and the diazirine group can be activated by light then form the covalent linkage with interacting proteins for capturing the proteins that can interact with PTMs. In the invention, the PEG passivation layer could help to reduce the non-specific adsorption of protein molecules, and the photo-affinity peptide probe could specifically recognize and capture the target protein molecule. The weak interaction between the photo-affinity peptide probe and the protein molecule is converted into strong covalent linkage under UV irradiation, thus achieving specific and efficient magnetic separation of the weak interacted protein molecule, ultimately, it provides a capture magnetic bead and capture approach for the study of weak protein-protein interactions. In addition, the formation and cleavage of disulfide bonds between NH2-PEG-SH and the photo-affinity peptide probe in the PEG-modified magnetic beads simplifies the capture step of protein molecules and facilitates the attachment of the photo-affinity probe to the magnetic beads, the release of the target protein molecules, and the reuse of capture magnetic beads.

The present invention is based on the preparation method and application of protein molecule capture magnetic beads by photo-affinity covalent linkage technology, said the magnetic bead is respectively modified by two functional molecular layers from the inside out, the polyethylene glycol PEG passivation layer and the photo-affinity peptide probe layer. The PEG passivation layer is used to reduce the non-specific adsorption of protein molecules, and the photo-affinity peptide probe can specifically recognize and capture the target protein molecule. The weak interaction between the photo-affinity peptide probe and the protein molecule is converted into strong covalent linkage under UV irradiation, thus achieving specific and efficient capture magnetic separation of the weak interacted protein, ultimately, it provides a capture magnetic bead and capture approach for the study of weak protein-protein interactions.

Beneficial effect: Compared with the current technology, the present invention has the following advantages:

The invention prepares a kind of capture magnetic bead that is able to targeting week protein-protein interactions, which enables the conversion of low-affinity interactions to strong and permanent covalent linkage, effectively achieving the capture of weak interaction protein molecules.

The photo-affinity magnetic beads (PAMBs) prepared by the invention ensure the capture of weakly interacting proteins with high spatial-temporal resolution and 81% reduction in non-specific interactions.

The photo-affinity magnetic beads (PAMBs) prepared by the invention have fewer interacting proteins and more protein sub-classes than conventional protein capture systems, indicating that the method has high specificity for the detection of PPIs mediated by PTMs.

(4) The photo-affinity magnetic beads (PAMBs) prepared by the invention contribute to the traditional method of studying protein-protein interactions and are suitable for the capture of weakly interacting protein molecules, while capture magnetic beads by the invention are simple to prepare, easy to use, time-saving, and have good specificity and resolution for the detection of weak and transient protein-protein interactions. The captured magnetic beads of the invention can fill the lack of the weak protein-protein interaction analysis technology, and are urgently needed in many biomedical research projects at present, and will have good application and market prospects.

### BRIEF DESCRIPTION OF DRAWINGS

FIG. 1 is a flow chart of the preparation of PAMBs.

FIG. 2 is a schematic diagram of the ligation amount and ligation efficiency analysis of peptide probes for PAMBs.

FIG. 3 is a schematic diagram of the characterization of the step-by-step preparation of PAMBs.

FIG. 4 is a schematic diagram of the feasibility analysis of PAMBs to capture interacting proteins.

FIG. 5 is a schematic diagram of the relationship between the capture amount of PAMBs interacting proteins and the sample protein concentration.

FIG. 6 is a schematic diagram of the relationship between the capture amount of PAMBs interacting proteins and the photo-affinity ligation time.

FIG. 7 is a schematic diagram of the performance differences between the photo-affinity system and the biotin-avidin system

Table 1 is a comparison of the performance of the photo-affinity system and the biotin-affinity system for capturing interacting proteins, with a table of mass spectrometry identification results.

### DETAILED DESCRIPTION

The present invention is further described below in conjunction with the accompanying drawings and examples.

The materials, reagents, etc. used in embodiments are available commercially if not otherwise specified.

The raw materials in the invention are commercially available.

Among them, NH2-PEG5000-SH was purchased from Ponsure, model PS2-SN-5K, dissolved in a buffer containing 50 mM borate, pH 8.5.

Magnetic beads were purchased from ThermoFisher, model 88826, as a solution of NHS-activated magnetic beads; 1 mL, magnetic beads dissolved in 10 mg mL⁻¹ N, N-dimethylacetamide DMAC.

### Example 1

The preparation method of PAMBs specifically comprises the following steps:

Synthesis of the peptide probe and synthesis of photo-sensitive part: The synthesis of the peptide was carried out by solid phase Fmoc method using Fmoc-Lys (Boc)-Wang resin (substitution value, 0.35 mmol g⁻¹) as the starting material, and synthesized from the C terminal to the N terminal. A volume ratio of 25% hexahydropyridine (dissolved in N,N′-dimethylformamide) was used to remove the N-terminal Fmoc protecting group and to make the N-terminus a free amino group, and then the amino acid raw material of 3 times the volume of resin is introduce into the second end of the C-terminus. The individual amino acid residues were repeatedly linked in turn to complete the synthesis of the entire peptide. The peptide with diazirine label was achieved by direct reaction of the diazirine-activated ester with a specific site lysine side chain amino group. After finishing each step of the above reaction, the resin was washed with DMF more than six times and the reaction was controlled by Kaiser Test. If a condensation reaction of an amino acid was incomplete, the condensation would be repeated once until the desired target peptide was obtained. Since diazirine was unstable under light, subsequent operations must be carried out under dark conditions. The target peptide was cleaved from the resin using with 90% by volume trifluoroacetic acid (TFA) cleavage reagent and removed the side chain protecting group at 30° C. for 3 h. Add a large amount of pre-cooled anhydrous ether to the filtrate and centrifuge to precipitate the peptide. After washing multiple times with diethyl ether, it was dried to obtain a crude peptide. The crude peptide was purified by reversed-phase high performance liquid chromatography (HPLC). Mobile phase A was aqueous solution containing 0.05% TFA acid and 2% acetonitrile (CAN), mobile phase B was 90% acetonitrile/water, flow rate was 25 mL min⁻¹. After lyophilizing the solvent, a pure peptide in a fluffy state was obtained, which is a photo-affinity peptide probe. The purity was determined by MALDI-TOF. Cysteine is an amino acid containing the thiol group.

The photo-affinity peptide probe synthesized in this Example is the tail peptide of Flap endonuclease 1 protein (FEN1), and the sequence of the photo-affinity peptide probe is shown below:
P1: C-K (Diazirine)-KAKTGAAGKFKRGK (SEQ ID NO.: 02);
P2: C-K (Diazirine)-KAKTGAAGKFKR (Me2a) GK (SEQ ID NO.: 01).

(2) Fixation and block: Specific method: The NH2-PEG5000-SH solution (PS2-SN-5K, pH 8.5) and magnetic beads (88826) was equilibrated to room temperature. Specifically, the magnetic beads were placed on the magnetic stand and discarded the supernatant, then gently vortexed for 15 s with adding pre-cooled 1 mL Wash Buffer A solution, collected the beads and immediately proceeded by adding 300 µL 50 mM NH2-PEG5000-SH solution, and then 30 s vortex oscillation. The solution was incubated with rotation for 2 h at room temperature. Magnetic beads were collected after washing two times with 1 mL 0.1 M glycine (pH 2.0) and then by ultrapure water, to obtain PEG-modified magnetic beads.

(3) The ligation of peptide: 0.5 mg mL⁻¹ photo-affinity peptide probe solutions P1 and P2 (40% DMSO aqueous solution as solvent) were prepared respectively, took 300 µL were immediately mixed into all the PEG-modified magnetic beads obtained in step 2 and mixed well. The mixtures were incubated with Shaker at 200 rpm for 4 h at room temperature. After adding 1 mL of Storage Buffer and mix well, we collected the beads on a magnetic stand, discarded the supernatant, and washed twice. Add 300 µL Storage Buffer and store the obtained capture magnetic beads (PAMBs) at 4° C. for later use.

Wash Buffer A above is 1 mM glacial acetic acid; Coupling Buffer is 50 mM borate pH 8.5 and 50 mM PB; NH2 -PEG5000-SH solution is the reagent with 0.1 M concentration; Wash Buffer B is 0.1 M glycine pH 2.0; Storage Buffer is Coupling Buffer with 0.05% sodium azide.

The preparation process is shown in FIG. 1, the prepared PAMBs contain several components that together perform the function of capturing protein. Firstly, at the surface of a magnetic bead, a PEG passivation layer is introduced to avoid nonspecific adsorption of proteins. Then, the photo-affinity peptide probe is introduced. There are two reactive groups at the N-terminal of the peptide probe, the thiol group on the side chain of cysteine (C) and the photo-reactive group (diazirine) modified on the ε-NH₂ of the lysine (K). The thiol group can link to the PEG layer by forming disulfide bond, and the diazirine group can be activated by light then form the covalent linkage with PTM-interacted proteins for capturing the proteins that can interact with PTMs. These two groups act together to facilitate the function of PAMBs in the capture system.

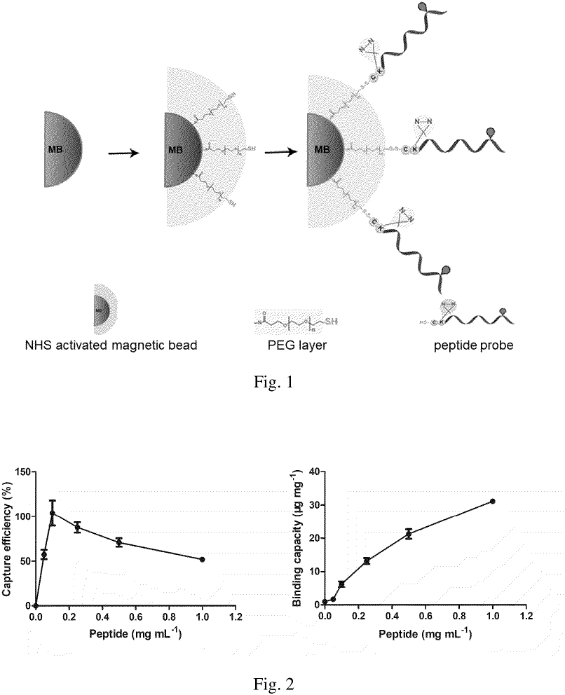

The analysis of the ligation amount and the conjunction efficiency of peptide probes in PAMBs as shown in FIG. 2. Conditions have been optimized to improve the performance of PAMB. Firstly, PAMBs were fabricated by using concentrations of 0, 0.05, 0.1, 0.25, 0.5, 1 mg mL⁻¹ peptide. With the increased concentration of peptide, the peptide probe attached to the magnetic beads has increased from 0.8 to 31.1 µg per mg of beads, and the conjunction efficiency has decreased to 51.9%, indicating that peptide probes attached to the magnetic beads surface were close to saturation. So, the peptide probe of 0.5 mg mL⁻¹ was used to prepare, and then ligation efficiency was 55.3%.

### Example 2

The weak protein-protein interaction capture magnetic bead based on photo-affinity covalent linkage for capturing weak interaction protein molecules.

Nuclear proteins were extracted by kits (CW0199S, Nuclear and Cytoplasmic Extraction Kit). Taking the nuclear protein of breast cancer MCF - 7 cells as an example, it was purified by protein purification column, and the protein concentration was adjusted to 1 mg mL ⁻¹. Adding 30 µL 0.5 mg mL⁻¹ PAMBs to 300 µL, incubated the solution at room temperature with shaking for 30 min, at the same time irradiated with a 365 nm UV lamp for 2 h. The magnetic beads were collected on the magnetic stand and discarded the supernatant, then washed the beads twice with PBST (0.05% Tween 20) and ultrapure water respectively, and collected the magnetic beads, the protein molecule linked to the beads is the captured protein molecule. Then the reactions were quenched by adding 30 µL 2x loading buffer (Beyotime), and the captured protein was released.

### Example 3

### Capture and Analysis of Arginine Methylated Proteins

The preparation method of PAMBs is the same as that of Example 1.

The terminal peptide of Flap Endonuclease 1 protein (FEN1) was used as peptide probe.

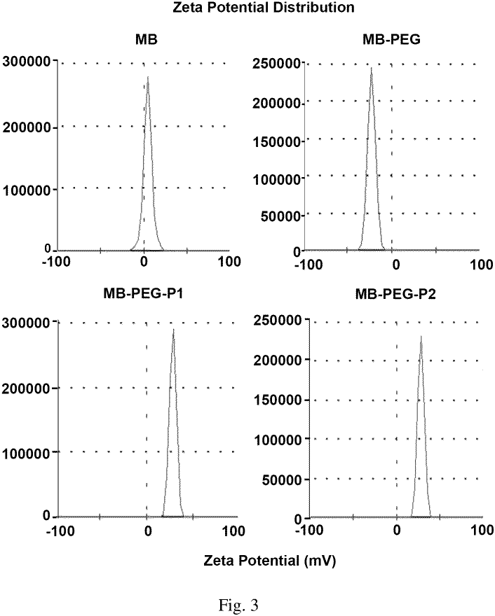

During the preparation of captured magnetic beads, PAMBs were respectively prepared by using unmethylated Flap endonuclease 1 (peptide probe P1) and methylated Flap endonuclease 1 (peptide probe P2). The PAMBs capture cellular nuclear protein assay was performed as in Example 2. The reactions were quenched by adding 30 µL 2x loading buffer (the lysis buffer of Beyotime), and the captured protein was released. Linking the peptide to the magnetic beads causes changes in the size and charge of the hydrated particles of the beads, so DLS and Zeta potential (DelsaNano C, USA) measurements was used to characterize the magnetic beads. As shown in FIG. 3, the Zeta potential of captured magnetic beads is 4.34 mV, while the potential significantly shifts to -22.5 mV after modification with a PEG passivation layer. The attachment of peptide probes can reverse the potential to 28.4 mV and 28.3 mV, respectively with further modification of MB-PEG-P1 and MB-PEG-P2. Slight Zeta-potential changes between MB-PEG-P1 and MB-PEG-P2 was caused by the methylation modification on the peptide probe P2, which indicate that the peptide is successfully attached to the magnetic beads.

### PAMBs Capture Methylation-Interacting Proteins

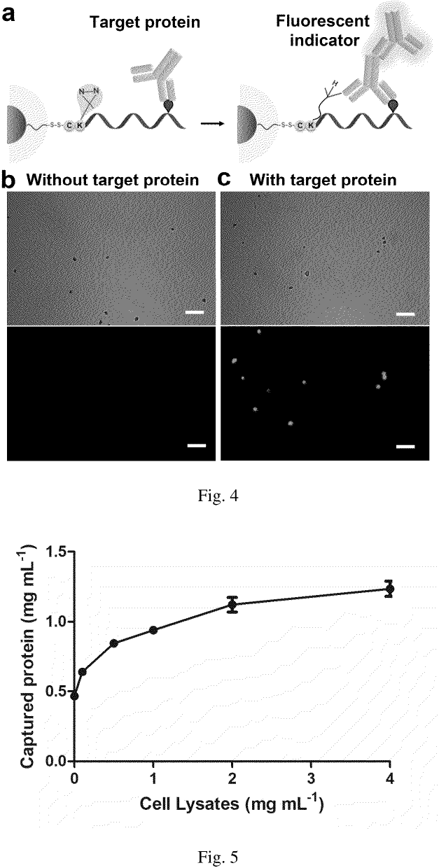

To test the feasibility of this assay, P1 and P2 were used as peptide probe, a PTMs site-specific (Rme2a) antibody was used as a capturable model protein(primary antibody, antibody concentration 1 mg mL⁻¹, diluted to 5 µg mL⁻¹ with 3% BSA), and a fluorescently labeled antibody (FITC-labelled IgG, antibody concentration 1 mg mL⁻¹, diluted to 5 µg mL⁻¹ with 3% BSA) as secondary antibody. Firstly, 200 µL PAMBs were incubated with 1 µL 5 µg mL⁻¹ primary antibody on a shaker at room temperature for 2 h, placed on a magnetic stand, discarded supernatant, and washed three times with PBST on a shaker for 5 min each. After the photo-affinity capture reaction, 200 µL 5 µg mL⁻¹ secondary antibody was added and incubated at room temperature for 1 h, placed on a magnetic stand, discarded supernatant, and washed three times with PBST on a shaker for 10 min each, discarded the supernatant. The ultrapure water was added to resuspend the magnetic beads, then 10 µL was placed on a slide and observed the fluorescence was observed under a microscope. The peptide probe P1 was unmethylated and could not capture the model protein primary antibody and could not bind to the fluorescently labeled antibody, so the fluorescence of the peptide probe P1 could hardly be observed, and the peptide probe P2 was methylated and fluorescence could be observed. FIG. 4 shows the microscopic images of PAMBs with and without captured model proteins, respectively. The fluorescence observation indicates that the photo-affinity capture magnetic bead can capture the proteins interacted by PTMs.

### Relationship Between the Capture Amount of PAMBs Interacting Protein and the Sample Protein Concentration

The PAMBs capture protein assay was performed as in Example 2. We used an enzyme marker at 560 nm to detect the capture protein concentration, and then tested the protein loading capacity of PAMBs. The amount of captured protein gradually increased as the cellular protein concentration increased, as in FIG. 5.

### Relationship Between the Capture Amount of PAMBs Interacting Protein and the Time of Photo-Affinity Attachment

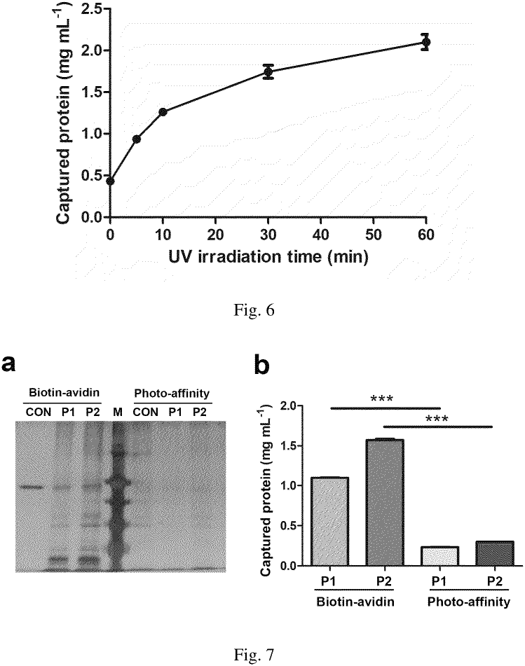

The PAMBs capture protein assay was performed as in Example 2. The protein quantification was performed to determine the concentration of captured protein (enzyme-labeled instrument 560 nm). With the prolongation of UV irradiation time, the amount of loaded protein on PAMB increased, and the maximum amount could reach 0.46 µg protein per mg of beads, indicating relatively high loading capacity of PAMBs, as in FIG. 6.

### Comparison of Performance Differences Between Photo-Affinity Capture Magnetic Beads and Biotin-Avidin Technology

1) Polyacrylamide gel method: The PAMBs capture protein assay was performed as in Example 2, and then the samples were heated at 95° C. for 5 min, the captured proteins were analyzed by polyacrylamide gel method with using P1 and P2 as peptide probes. Biotin-avidin technique and PAMBs were used for protein capture experiments, respectively. The polyacrylamide gels showed significantly less amount of protein captured by photo-affinity than biotin-avidin, as shown in the upper panel of FIG. 7; biotin-avidin was used by existing common methods.
2) Protein quantification assay: Protein quantification assay of the captured protein concentration (enzyme-labeled instrument 560 nm) showed a significant reduction in the amount of photo-affinity captured proteins, the reduction of P1 and P2 of the photoaffinity capture magnetic beads was 81.7% and 80.7%,, as shown in the lower panel of FIG. 7.

### Comparison of the Mass Spectrometric Analysis of Photo-Affinity Capture Magnetic Beads and Biotin-Avidin Capture Interaction Proteins

Proteins were separated by polyacrylamide gel, experimental method as in (5) of Example 3, using Pierce Silver Stain Kit (24612, Thermo) to dye. The gel was washed for 5 min in ultrapure water, then fixed in 30% ethanol containing 10% acetic acid solution for 15 min, a total of two times. Washed gel in 10% ethanol, then in ultrapure water twice for 5 min each time, added sensitizer working solution (50 µL sensitizer with 25 mL water) . After 1 min, the gel was washed with water twice for 1 min each time. The gel stained in stain working solution (0.5 mL enhancer with 25 mL stain solution) for 30 min, washed twice with ultrapure water for 20 s each time., then developed in Developer Working Solution (0.5 mL enhancer with 25 mL developer solution) until bands appeared. The color reaction was terminated by 5% acetic acid for 10 min. The gel containing the specific band was cut into 1 mm wide cubes and then destained by incubation with 50 mM ammonium bicarbonate and 50% acetonitrile for 1 h. The destained gel was lyophilized and dehydrated, and the protein was digested by trypsin overnight at 37° C. Peptides were collected and dried, then dissolved in 0.5% acetic acid for analysis by LC/MS/MS. Photo-affinity capture magnetic beads had fewer and more types of proteins. Besides, it is noted that about 60% of the low abundant proteins can be captured, much higher than 16% in the biotin-avidin system. The results may indicate that photo-affinity capture magnetic beads were less interfered by the over expressed proteins in the sample. Moreover, the photo-affinity capture magnetic beads showed the interaction was specific when compared to the biotin-avidin system, indicating high specificity of the developed system for PTM-mediated PPIs, as is shown in Table1, further indicating that traditional methods for identifying protein interactions are subject to non-specific adsorption and that the capture magnetic bead can capture fewer proteins, reducing non-specific binding.

TABLE 1

| Biotin-Avidin system | | | Photo-Affinity system | |
|---|---|---|---|---|
| Classify | Quantity | | Classify | Quantity |
| Cytoskeleton | | 5 | Cytoskeleton | 4 |
| RNA | Ribosomal | 14 | RNA | Ribosomal 0 |
| | RNA binding | 6 | | RNA binding 4 RNA helicase 1 Splicing factor 1 Translation 1 |
| Histone | | 3 | DNA damage Oxidation ATP synthesis | 1 1 2 |
| Others | | 10 | Others | 3 |
| Total | | 38 | | 18 |

What is claimed is:

1. A capture magnetic bead for targeting weak protein-protein interactions with photo-affinity covalent linkage, wherein the magnetic bead is modified by a polyethylene glycol (PEG) passivation layer and a photo-affinity peptide probe layer, wherein the PEG passivation layer is introduced at the surface of a magnetic bead, forming a PEG-modified magnetic bead, and the photo-affinity peptide probe layer is a molecular layer of peptide whose N-terminal end being modified with a thiol group and a diazirine group.

2. The capture magnetic bead according to claim 1, wherein a PEG in the PEG passivation layer is a PEG molecule respectively modified with the amino group and the thiol group at each end, respectively; the PEG molecule is NH2-PEG-SH that is served as an intermediate linking molecule to connect magnetic beads and photo-affinity peptide probes.

3. The capture magnetic bead according to claim 1, wherein the photo-affinity peptide probe layer is a synthesized peptide whose N-terminal end being modified with the thiol group and the diazirine group; the thiol group is modified on side chain of cysteine and the diazirine group is modified on ε-NH₂ of lysine.

4. The capture magnetic bead according to claim 3, wherein the sequence of the synthesized peptide is a modified cysteine (Cys) at N-terminal, followed by a diazirine modified lysine (Lys), followed by a peptide capable of targeting interacted proteins, peptide pattern sequence is CK (Diazirine)(X)ₙ, wherein X represents amino acids or post-translational modification amino acids, n is an integer.

5. A process for preparing the capture magnetic bead of claim 1 comprising the following steps:
i) adding a solution of NH₂-PEG-SH into magnetic beads at room temperature and mixing; then separating the magnetic beads as PEG-modified magnetic beads;
ii) linking covalently carboxyl groups of C-terminal of a peptide with an insoluble polymer resin, deprotecting the C-terminal to the N-terminal by removing protective group on α-amino group of amino acid; then activating, coupling, washing and filtrating; labeling the peptide with diazirine by reacting succinimidyl ester-activated diazirine with amino group of a lysine side chain at N-terminal of the peptide; obtaining photo-affinity peptide probes by cleaving the peptide from the insoluble polymer resin; and
iii) Preparation of photo-affinity capture magnetic beads: mixing and incubating the photo-affinity peptide probes and PEG-modified magnetic beads at room temperature; adding dimethyl sulfoxide which linking the photo-affinity peptide probes to the magnetic beads by disulfide bonds; obtaining the capture magnetic bead after washing with a phosphate buffer solution.

6. The process according to claim 5, wherein in the step i), the solution of NH₂-PEG-SH and the magnetic beads are equilibrated to room temperature, the magnetic beads are placed on a magnetic stand and discarding the supernatant, then gently vortexing after adding glacial acetic acid solution, collecting the magnetic beads and adding the NH₂-PEG-SH solution immediately, then vortexing, incubating with rotation at room temperature, collecting the PEG-modified beads after washing, and the PEG-modified beads at 4° C.

7. A method for utilizing the capture magnetic bead of claim 1 comprising a step of adding capture magnetic beads into a sample.

8. The method according to claim 7, wherein the method further comprising: adding the capture magnetic beads into the sample, vortexing gently at room temperature, and simultaneously irradiating with an ultraviolet lamp to activate covalent linkage between diazirine and captured protein, then collecting and washing the capture magnetic beads, proteins linked to the capture magnetic beads are captured target proteins, adding reducing reagent to cleave disulfide bonds between NH₂-PEG-SH and the photo-affinity peptide probe, releasing the target proteins for further analysis with polyacrylamide gel and mass spectrometry.
